(19) United States
(12) Patent Application Publication
(10) Pub. No.: US 2009/0149858 A1
(43) Pub. Date: Jun. 11, 2009

(54) Method And Apparatus For Forming A Bone Tunnel

(75) Inventors: Gregory Fanelli, Danville, PA (US); Stephen M. Howell, Elk Grove, CA (US); Ryan A. Kaiser, Leesburg, IN (US)

Correspondence Address: HARNESS, DICKEY & PIERCE, P.L.C., P.O. BOX 828, BLOOMFIELD HILLS, MI, 48303, US

(73) Assignee: BIOMET SPORTS MEDICINE, INC., Warsaw, IN (US)

(21) Appl. No.: 11/950,550

(22) Filed: Dec. 5, 2007

(51) Int. Cl. A61B 17/00; A61B 17/58

(52) U.S. Cl. 606/80; 606/96

(57) ABSTRACT

A tool and method for drilling a bone for receipt of a first and second bundle includes drilling a first tunnel in the bone. A locating instrument may be positioned into the first bone tunnel thereby defining a reference axis. A tool may be inserted through a portal defined in surrounding tissue. The tool may engage the locating instrument thereby identifying a relative orientation between the reference axis and the drill axis for preparation of a second bone tunnel. The second bone tunnel may be drilled along the drill axis.

### FIELD

This invention relates generally to a method and apparatus for use in ligament reconstruction, and more specifically to a method and apparatus for drilling a second tunnel, relative to an already prepared first tunnel.

### BACKGROUND

The knee joint is frequently the object of injury and is often repaired using arthroscopic surgical procedures. Numerous improvements in repairing damage to knee joints have been made over the years, and some of the major advances involve the use of endoscopic techniques and arthroscopic procedures. Arthroscopic surgery may be particularly useful in excising or repairing damaged knee cartilage.

Endoscopic techniques have also been developed for use in repair and reconstruction of damaged anterior cruciate ligaments (ACL) and posterior cruciate ligaments (PCL). When the ACL in particular has ruptured and is non-repairable, it may be replaced in young adults and the knee reconstructed through the use of grafts. While not as common, the PCL may alternatively or additionally be replaced.

The function of the real cruciate ligaments is complicated. The ACL and PCL are three-dimensional structures with broad attachments and a continuum of fibers. These fibers are of different lengths, have different attachments sites, and are under different tensions.

One approach to ACL reconstruction is replacing the ACL with a dual-bundle ACL replacement. More specifically, an anterior medial (AM) tunnel may be prepared for receipt of an AM bundle and a posterior lateral (PL) tunnel may be prepared for receipt of a PL bundle. In many instances however, relative tunnel placement between the AM tunnel and PL tunnel may be difficult.

As with ACL reconstruction, one approach to PCL reconstruction is replacing the PCL with a dual-bundle PCL replacement. For PCL replacement, a posterior medial (PM) tunnel may be prepared for receipt of a PM bundle and an anterior lateral (AL) tunnel may be prepared for receipt of an AL bundle. Sometimes however, relative tunnel placement between the PM tunnel and the AL tunnel may be difficult.

### SUMMARY

A tool for use during dual-bundle ligament reconstruction can include a body having a distal portion and a proximal portion. The body can define a cannulated longitudinal shaft defining a first axis. A locating feature such as a groove can be formed in the distal portion defining a second axis arranged at a predetermined orientation relative to the first axis. A first locating instrument can be extended through a first bone tunnel and adapted to rest against the locating surface of the groove. The first axis can operatively align at the predetermined orientation thereby identifying a drill axis for preparation of a second bone tunnel.

According to additional features, the distal portion can include a distal engaging surface oriented at a non-orthogonal angle relative to the first axis. The proximal portion can include a handle defining a second axis oriented at a non-orthogonal angle relative to the first axis. In one example, the longitudinal shaft can define a first outer diameter and the distal portion defines a second outer diameter, the first outer diameter being less than the second outer diameter.

A tool according to other features of the present teachings includes a first locating instrument defining a first longitudinal shaft portion having a first axis, a first reference head and a first distal tip portion. A second locating instrument defines a second longitudinal shaft portion having a second axis and a second reference head. The first locating instrument can be adapted to be located into a first bone tunnel. The second reference head of the second locating member can be adapted to engage the first reference head to operatively align the second axis at a predetermined orientation thereby identifying a drill axis for preparation of a second bone tunnel.

A method for drilling a bone for receipt of a first and second bundle includes drilling a first tunnel in the bone. A locating instrument can be positioned into the first bone tunnel thereby defining a reference axis. A tool can be inserted through a portal defined in surrounding tissue. The tool can engage the locating instrument thereby identifying a relative orientation between the reference axis and the drill axis for preparation of a second bone tunnel. The second bone tunnel can be drilled along the drill axis.

Further areas of applicability of the present disclosure will become apparent from the detailed description provided hereinafter. It should be understood that the detailed description and various examples, while indicating various embodiments of the invention, are intended for purposes of illustration only and are not intended to limit the scope of the following claims.

### BRIEF DESCRIPTION OF THE FIGURES

The present invention will become more fully understood from the detailed description and the accompanying drawings, wherein:

FIG. 1A is a lateral view of a tibia and femur in phantom shown with an exemplary femoral aimer inserted through a tibial tunnel and a graft passing pin drilled through the lateral thigh according to an exemplary method;

FIG. 2 is an anterior view of the knee illustrated in FIG. 1 shown with a calibrated acorn reamer used to determine the length of the femoral tunnel;

FIG. 3 is a side view of an exemplary tool for drilling a second tunnel relative to a first tunnel according to the present teachings;

FIG. 4 is a sectional view taken along the longitudinal axis of the tool of FIG. 3;

FIGS. 5A-5C illustrate exemplary distal end portions of the tool shown in FIG. 3;

FIG. 6 is a detailed perspective view of the distal end portion of FIG. 5A;

FIG. 7A is a top view of the distal end portion of FIG. 5A;

FIG. 7B is an axial end view of the distal end portion of FIG. 5A;

FIG. 8 is a detailed perspective view of the distal end portion of FIG. 5B;

FIG. 9 is an anterior view of a right knee illustrating the tool of FIG. 3 engaging a locating member extending through a first bone tunnel (anterior medial tunnel) to determine the location of a second bone tunnel (posterior lateral tunnel) during dual bundle ACL reconstruction;

FIG. 10 illustrates a cannulated drill preparing the first and second bone tunnels;

FIG. 11 illustrates anterior medial (AM) and posterior lateral (PL) bundles secured by anchors in respective AM and PL tunnels according to an exemplary method;

FIG. 12 is an exploded view of an exemplary tool according to additional features of the present teachings;

FIG. 13 is an anterior view of a right knee shown with one tool of FIG. 12 positioned in a first bone tunnel (anterior lateral tunnel) and a second tool of FIG. 12 used to determine the location of a second bone tunnel (posterior medial tunnel) during dual bundle PCL reconstruction;

FIG. 14 illustrates anterior lateral (AL) and posterior medial (PM) bundles secured by anchors in respective AL and PM tunnels according to an exemplary method; and

FIG. 15 illustrates anterior lateral (AL) and posterior medial (PM) bundles secured by anchors in respective AL and PM tunnels according to another exemplary method.

### DETAILED DESCRIPTION OF VARIOUS EMBODIMENTS

The following description of various embodiment(s) is merely exemplary in nature and is in no way intended to limit the application or uses. Those skilled in the art will appreciate that the following teachings can be used in a much wider variety of applications than the examples specifically mentioned herein. More specifically, while the following discussion is specifically directed toward a tool and method for dual bundle ACL and/or PCL reconstruction, the same may be applied to other surgical procedures where locating a second tunnel relative to a first tunnel in bone is desired.

At the outset, an exemplary procedure for preparing a tibial tunnel and a femoral tunnel will be described. For a knee reconstruction involving an un-repairable or torn ACL, the procedure may begin with a general anesthesia being administered to the patient. The patient may be positioned supine on an operating table. A well-padded tourniquet may be placed proximal on the thigh of the affected leg. An arthroscopic leg holder may be placed around the tourniquet. The table may be inclined (e.g. 15 degrees of trendelenburg) and adjusted in height (e.g. waist level) according to the desires of the surgeon. The other leg may be secured to the foot of the table. A Mayo stand may be placed over the leg holder and positioned to permit access to the lateral thigh.

The surgical site may be prepped and draped with a sterile seal. Standard arthroscopic draping may be performed covering the Mayo stand. A light cord, camera, motorized instruments, inflow, outflow and suction tubing may be wrapped and secured to the drape on the Mayo stand. An irrigation stand may be set up and positioned. The joint of the affected leg may be examined physically to confirm a rupture of the ACL and to determine the amount and degree of movement.

The graft harvesting step in the procedure depends on the type of ACL substitute that is to be utilized. In one example, the gracilis and semi-tendinosus tendons are harvested from the patient and used as the ACL substitute. In general these may provide grafts which are stronger in the joint than the original ACL and have less postoperative morbidity. In other examples, patellar tendons, autogenous tendons, frozen and lyophilized tendon allografts, or some of the various known synthetic materials may be used as ACL substitute.

After the grafts are harvested, they may be prepared and sized. In one example, a surgical assistant may prepare and size the grafts while the surgeon continues with the rest of the ACL replacement procedure. Sutures may be attached to the ends of the grafts to aid in grasping, manipulating and securing the grafts in place. Incremental sizing tubes may be used to size the grafts and select the appropriate drills for forming the tunnels. For purposes of discussion, the ACL grafts will hereinafter be referred to as an anterior medial (AM) bundle and a posterior lateral (PL) bundle.

The prepared knee may now be examined by arthroscopic procedures. Standard anterolateral and anteromedial portals may be made for the diagnostic arthroscopy. Proper portal placement is important. In one example, the lateral portal may be made at a location one-third the width of the patella ligament medial to the lateral margin and positioned vertically just inferior to the inferior patella tip. The medial portal may be made vertically, just inferior to the inferior patella tip and adjacent to the medial border of the patella ligament. The two portals may be located at the same level.

In one example, wallplasty may be performed to remove a portion of the lateral condylar wall. A tool such as an up-angled, curved and uterine curette may be used to remove the origin (and stump) of the ACL from the intercondylar roof and the wall of the lateral femoral condyle. The retained synovial and cruciate remnants may be cleaned and vacuumed with a full-radius resector.

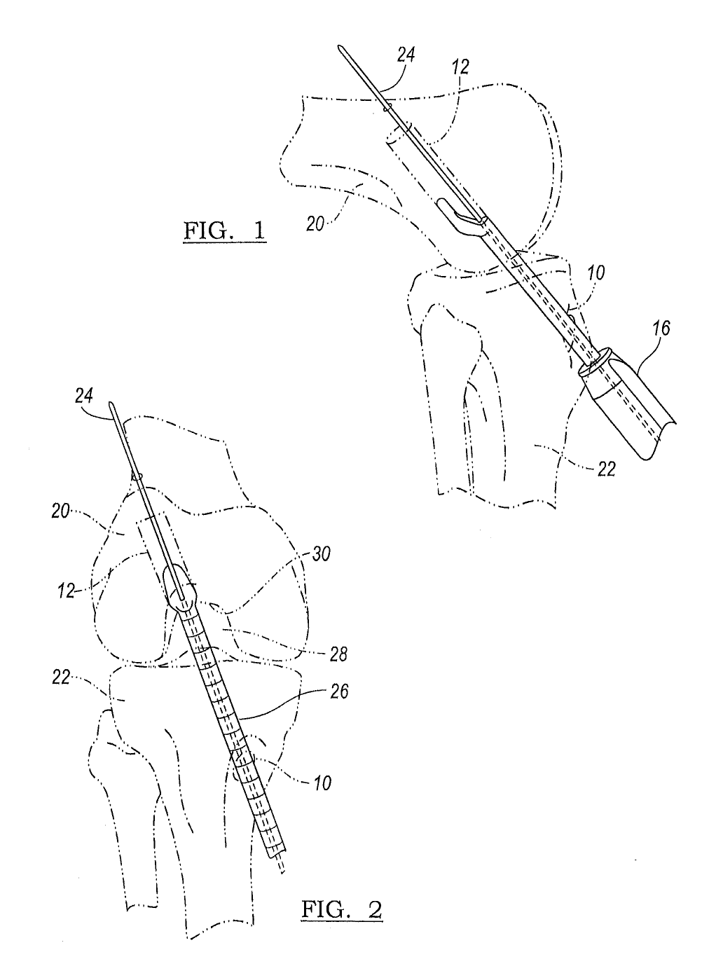

Next, a tibial tunnel 10 (FIG. 1) is prepared. In one example, a HowelI™ 65° Tibial Guide (Howell Guide, not shown) may be used to prepare the tibial tunnel 10. The Howell Guide and a method of using may be found in U.S. Pat. No. 6,254,605, which is incorporated by reference.

With reference now to FIGS. 1 and 2, an exemplary method of preparing the femoral AM tunnel 12 will be described. Once the tibial tunnel 10 has been prepared, a femoral aimer 16 may be inserted through the tibial tunnel 10. In one example, a 12 mm femoral aimer 16 may be used. The femur 20 may then be flexed to 90° relative to the tibia 22. The femoral aimer 16 may then be laterally angulated and externally rotated away from the PCL (not shown). A graft passing pin 24 may then be drilled through the lateral thigh. The length of the femoral AM tunnel 12 may be equal to the length of the AM graft minus the length of the tibial tunnel 10 and notch (FIG. 2). A calibrated acorn reamer 26 may be used to measure the length of the tibial tunnel and notch 28. The acorn reamer 26 may be inserted over the graft passing pin 24 until it touches the intercondylar roof 30. The length of the acorn reamer 26 at the point the reamer 26 exits the tibial tunnel 10 is recorded. The acorn reamer 26 may then be used to drill the femoral AM tunnel 12 to the calculated length.

One exemplary method of securing the replacement grafts to the respective AM and PL tunnels includes using interference screws such as a TunneLoc® Interference Screw manufactured by the assignee of the present disclosure. Although the following discussion will be directed toward utilizing interference screws, other methods may be used to secure the respective AM and PL bundles within the AM and PL tunnels. When preparing the AM and PL tunnels for interference screws, a tunnel notcher (not shown) may be used to notch the anterior rim of the AM and PL tunnels to facilitate improved contact with the interference screw.

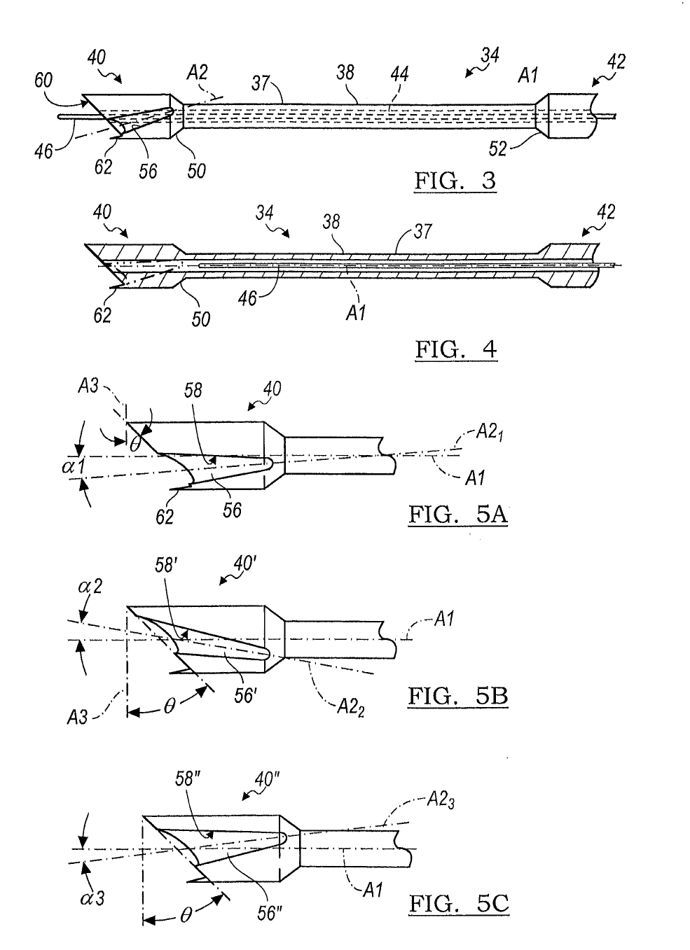

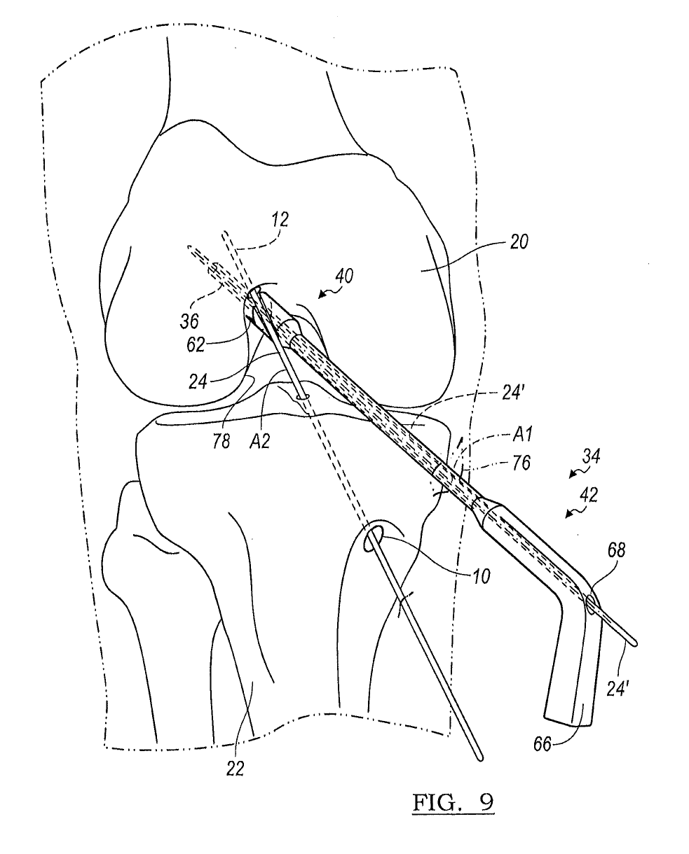

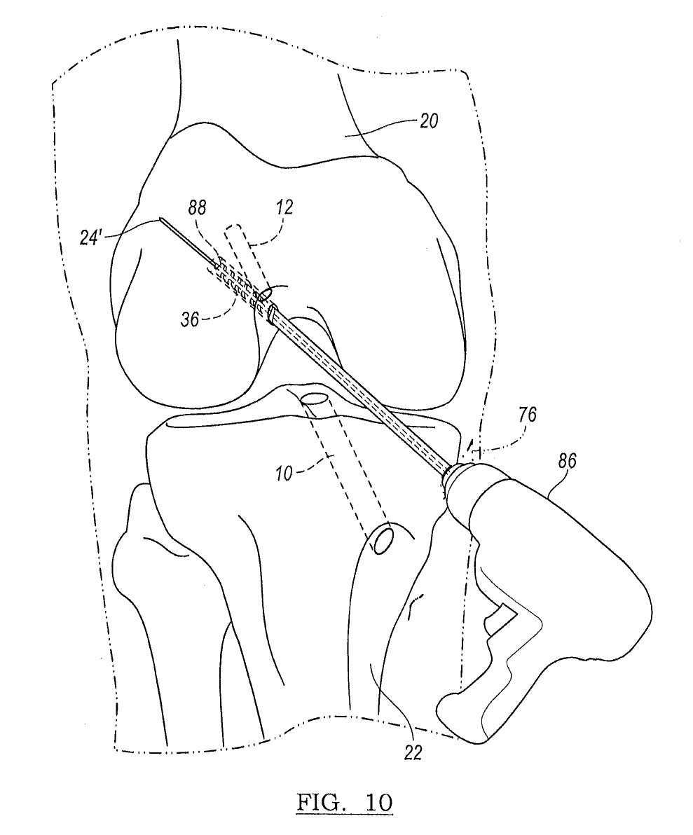

With reference now to FIGS. 3-8, an exemplary tool 34 for preparing a PL tunnel 36 (FIG. 9) relative to the AM tunnel 12 will be described. It is appreciated however, that although the following discussion is directed toward locating the PL tunnel 36 relative to the AM tunnel 12, the tool 34 may be used to locate any tunnel relative to an already prepared tunnel. For example, the tool 34 may be used to locate the AM tunnel 12 relative to an already prepared PL tunnel 36. In another example, the tool 34 may be used to locate one bone tunnel relative another bone tunnel for receipt of a dual bundle (anterolateral bundle and posteromedial bundle) posterior cruciate ligament (PCL) replacement.

The tool 34 generally includes a body 37 having a longitudinal shaft 38 extending between a distal portion 40 and a proximal portion 42. The longitudinal shaft 38 may define a cannulation 44 defining a first axis A1. The cannulation 44 may be adapted to accept a guide wire or second graft passing pin 46 therethrough for preparation of a bone tunnel as will be described in greater detail. The distal portion 40 may define an outer diameter greater than an outer diameter defined by the longitudinal shaft 38. Similarly, the proximal portion 42 may define an outer diameter greater than the outer diameter of the longitudinal shaft 38. In one example, respective tapered portions 50 and 52 are formed at the transition between the respective distal and proximal portions 40 and 42 and the longitudinal shaft 38.

The distal portion 40 will be described in greater detail. The distal portion 40 may define a locating feature such as a groove 56. The groove 56 includes a locating surface 58 defining a second axis A2. The second axis A2 is defined on the locating surface 58 at the deepest portion of the groove 56. As will be described in relation to FIG. 9, the tool 34 may be used to orient the cannulation 44 (first axis A1) relative to a locating member 24 positioned against the groove 56 (at axis A2) in a first bone tunnel. In this way, the position of a second bone tunnel (such as the PL tunnel 36) may be determined based on the position of an already formed bone tunnel such as the AM tunnel 12 (marked by the locating member 24 extending through the first bone tunnel). It is appreciated that the locating feature may define other configurations in addition to a groove.

Returning again to FIGS. 3-8, the distal portion 40 may define a distal engaging surface 60. The distal engaging surface 60 may define an angle θ relative to an axis A3 normal to the first axis A1. A spike 62 may be formed on the distal engaging surface 60 for facilitating gripping on bone (such as a medial or lateral condyle of the knee) during use. As will be described in relation to one example, the distal engaging surface 60 may be used to rest against a medial condyle of the knee during operation.

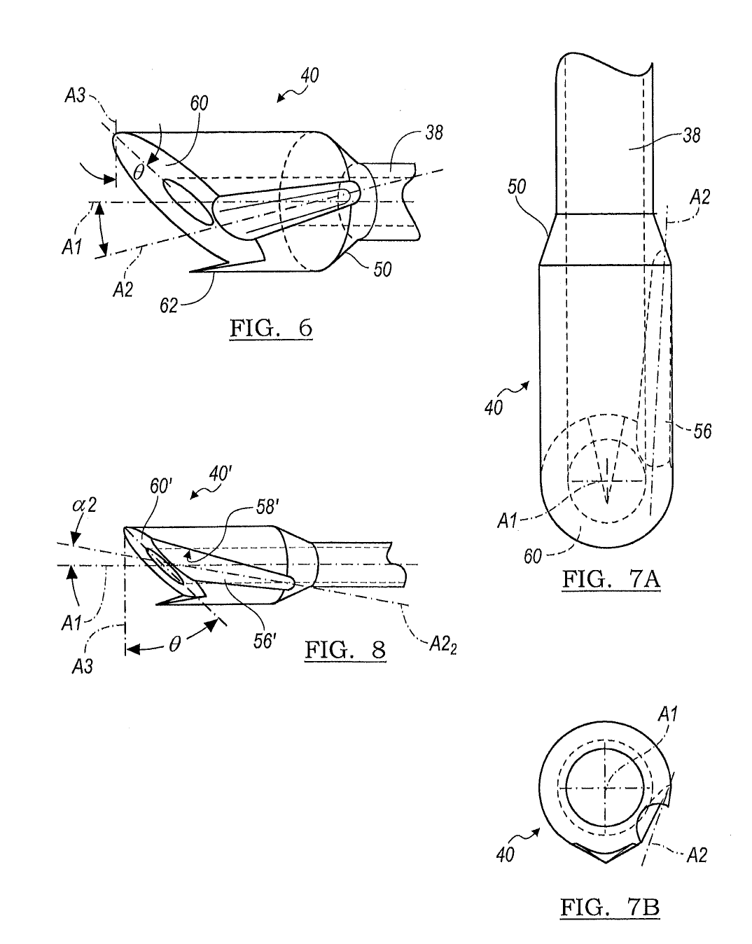

With specific reference now to FIGS. 5A-5C, a series of distal portions 40, 40′ and 40″ are shown each having a distinct groove 56, 56′ and 56″ and each defining a unique axis A2₁, A2₂, and A2₃. The distal portion 40 (FIG. 5A) provides a groove 56 defining an angle of α1 relative to axis A1. The distal portion 40′ (FIG. 5B) provides a groove 56′, defining an angle of α2 relative to axis A1. The distal portion 40 (FIG. 5C) provides a groove 56″ defining an angle of α3 relative to axis A3. In this way, a surgeon may select a tool having a predefined orientation of the axis A2 relative to axis A1 according to the particular needs of a given patient. While not specifically shown, the respective distal portions 40, 40′ and 40″ may be releasably connected to the longitudinal shaft 38 by way of threads or other connecting feature. It is appreciated that the distal portions 40, 40′ and 40″ may additionally or alternatively define distinct engaging surfaces for providing various angles θ. FIGS. 6-8B illustrate various views of the distal portion 40 of the tool 34.

With reference now to FIG. 9, the proximal portion 42 of the tool 34 will be described in greater detail. The proximal portion 42 may define a handle 66. The handle 66 may generally provide a gripping surface arranged at an angle with respect to the first axis A1. In this way, the handle 66 may be easily rotated during manipulation of the tool 34. A passage 68 is formed at a proximal end surface for accepting a guide wire 24′ prime and/or drill bit during use.

With continued reference to FIG. 9 and further reference to FIGS. 2 and 10, an exemplary method for using the tool 34 will be described in detail. Again, as described above, while the following description will be directed toward preparing a PL tunnel 36 (FIG. 7) relative to the AM tunnel 12, the tool 34 may be used to locate any tunnel relative to an already prepared tunnel. For example, the tool 34 may be used to locate the AM tunnel 12 relative to an already prepared PL tunnel 36. In another example, the tool 34 may be used to locate one bone tunnel relative another bone tunnel for receipt of a dual bundle (anterolateral bundle and posteromedial bundle) posterior cruciate ligament (PCL) replacement.

As shown in FIG. 9, a first locating instrument 24 is illustrated extending through the tibial tunnel 10 and into the AM tunnel 12. The first locating instrument 24 may comprise any rigid longitudinal member defining the axis A2. In this way, the first locating instrument 24 may include the graft passing pin, a k-wire, a drill bit or other longitudinal member.

Next, the tool 34 may be inserted through an AM portal 76 until the groove 56 defined in the distal portion 40 receives the first locating instrument 24 in an operating position as shown in FIG. 9. Concurrently, the distal engaging surface 60 may engage the lateral condyle 78 such that the spike 62 slightly pierces the lateral condyle 78 facilitating a secure position. It is appreciated that while inserting the tool 34 through the AM portal 76 to attain the operating position, it may be necessary to rotate the tool 34 about the axis A1 to properly align the groove 56 for receipt of the first locating member 24. The orientation of the handle may provide a surgeon a moment arm for convenient rotational manipulation of the tool 34 during alignment.

Once the tool 34 is in the operating position, the axis A1 defined by the cannulation 44 of the tool 34 is oriented at the desired position relative to the axis A2 of the first locating member 24. In this way, a surgeon may accurately predict the placement of the second bone tunnel (in this example, the PL tunnel 36). The guide wire or second graft passing pin 24′ may then be translated into the passage, through the cannulation 44 and into the newly formed PL tunnel 36.

With reference now to FIGS. 10, preparation of the PL tunnel 36 will be described according to one example. A drill 86 having a cannulated drill bit 88 may be used to drill the PL tunnel 36. The drill bit 88 may be inserted over the pin 24′ and then may be used to drill the femoral PL tunnel 36. It is appreciated that the PL tunnel may be prepared differently while utilizing the desired orientation provided by the tool 34.

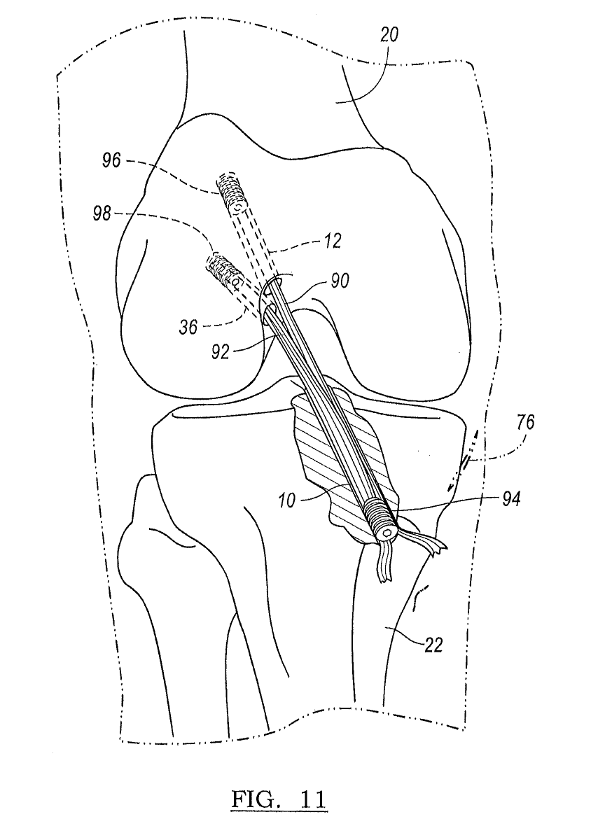

Turning now to FIG. 11, an exemplary method for securing an AM bundle 90 and a PL bundle 92 to the AM tunnel 12 and the PL tunnel 36, respectively will be described. Interference screws such as TunneLoc® Interference Screws 94, 96, and 98 may be driven by conventional methods into the tunnels 10, 12 and 36 respectively to securely locate the AM bundle 90 and PL bundle 92. While not specifically shown, in an alternate method, a unique tibial tunnel may be prepared specifically for the PL bundle 92. As a result, the AM bundle 90 and the PL bundle 92 would not need to share a common tibial tunnel.

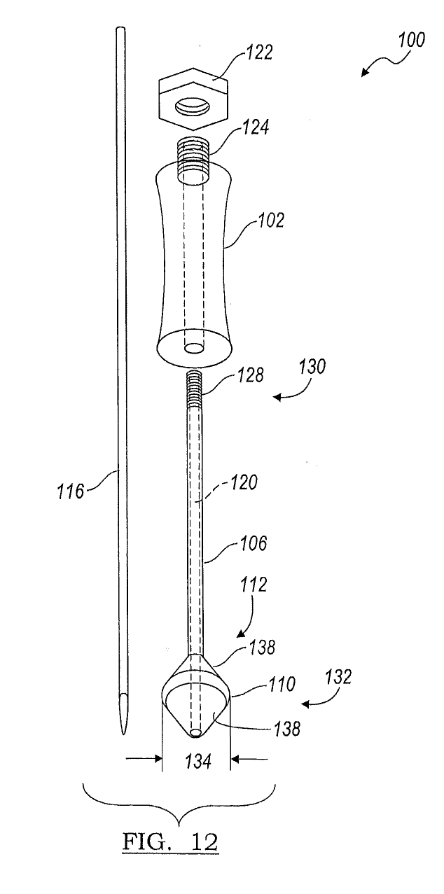

With reference now to FIGS. 12-15, an exemplary tool 100 for preparing a PM tunnel relative to an AL tunnel will be described. It is appreciated however, that although the following discussion is directed toward locating the PM tunnel relative to the AL tunnel, the tool may be used to locate any tunnel relative to an already prepared tunnel. For example, the tool 100 may be used to locate the AL tunnel relative to an already prepared PM tunnel. In another example, the tool 100 may be used to locate one bone tunnel relative to another bone tunnel for receipt of a dual bundle (AM bundle and PM bundle) anterior cruciate ligament (ACL) replacement.

The tool 100 generally includes an aimer handle 102, a guide 106 having a head 110 formed on a distal end 112 and a pin 116. The guide 106 may define a cannulation 120 for accepting the pin 116 therethrough. In one example, a nut 122 may threadably secure the aimer handle 102 to the guide 106. In one example, the aimer handle 102 may define threads 124 for mating with the nut 122. Alternatively, the nut 122 may threadably secure directly to threads 128 formed on a proximal portion 130 of the guide 106. The head 110 may generally define a central body 132 having an outer diameter 134 corresponding to a diameter of a desired endoscopic reamer. For example, the head 110 may define a diameter between 7 and 12 mm for example. The head 110 may define tapered portions 138 extending from the central body 132.

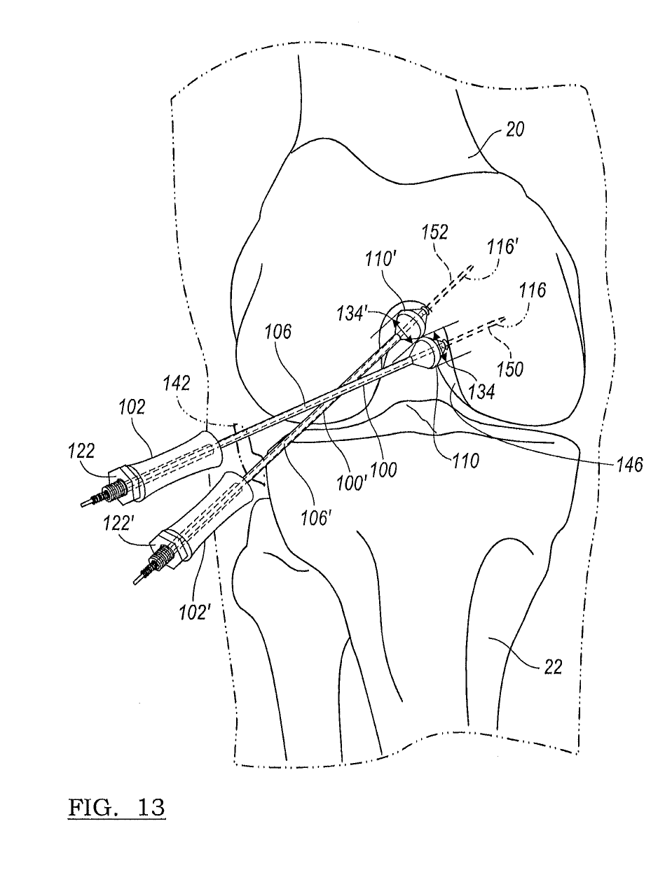

Turning now to FIG. 13, an exemplary method of using the tool 100 will be described. In general, a pair of tools 100 may be used to identify a relative orientation between each other. For clarity, a second tool will be referred to as 100′. As such, each feature of the second tool 100′ will be referred to with like reference numerals as the first tool 100 and be denoted by a prime suffix. As will be described, the pair of heads 110 and 110′ may engage each other to identify a tunnel offset such that the respective tunnel distances are a set distance apart at that point. In one example, an AL portal 142 may be formed to gain access to the medial condyle 146 of the knee.

At the outset, a first tool 100 is located into a position corresponding to the AL tunnel 150. In one example, the pin 116 may be inserted into a hole defining the orientation of an AL tunnel 150. The hole may define an already prepared AL tunnel 150, or a preliminary hole used to direct an endoscopic reamer for subsequent formation of the AL tunnel 150. Once the first tool 100 is located into the AL tunnel 150, the second tool 100′ may be passed through the AL portal 142 and manipulated until its head 110′ engages the head 110 of the first tool 100. Specifically, the outer diameter 134′ of the second head 110′ may be engaged to the outer diameter 134 of the first head 110 (FIG. 13). The surgeon may then manipulate the second tool 100′ relative to the first tool 100 until a desired orientation is achieved according to the specific needs of a patient. It is appreciated, that during such manipulation, contact between the respective heads 110 and 110′ verifies proper spacing between the AL tunnel 150 and a perspective PM tunnel 152. Once the second tool 100′ has attained a desired orientation, the pin 116′ may be passed through the guide 106′ thereby defining the PM tunnel placement relative to the AL tunnel 150. The PM tunnel 152 may then be reamed by conventional methods such as using an endoscopic reamer.

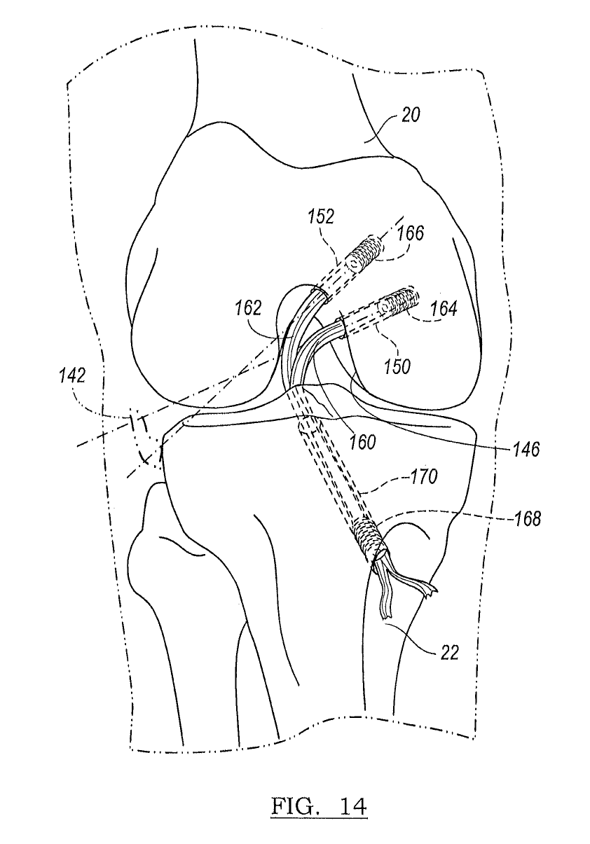

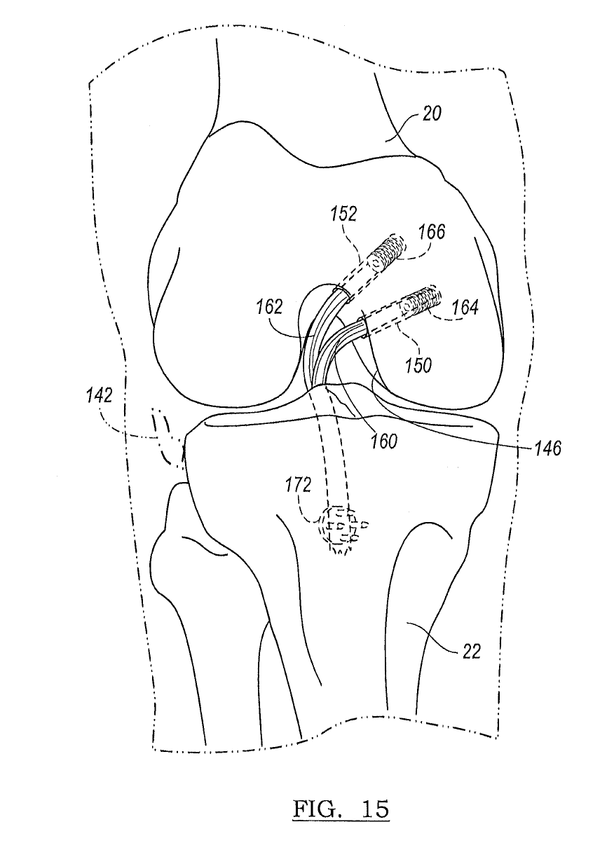

Turning now to FIGS. 14 and 15, exemplary methods for securing an AL bundle 160 and a PM bundle 162 to the AL tunnel and the PM tunnel 150 and 152, respectively will be described. Interference screws such as TunneLoc® Interference Screws 164, 166 may be driven by conventional methods into the tunnels 150 and 152, respectively to securely locate the AL bundle 160 and the PM bundle 162. As illustrated in FIG. 14, another TunneLoc® Interference Screw 168 may be driven into a PM portal 170 formed through the tibia for accepting the respective bundles 160 and 162. Alternatively, as shown in FIG. 15, the respective bundles 160 and 162 may be secured directly to the anterior tibia 22 such as by a bone screw 172.

Those skilled in the art can now appreciate from the foregoing description that the broad teachings of the present invention can be implemented in a variety of forms. Therefore, while this invention has been described in connection with particular examples thereof, the true scope of the invention should not be so limited since other modifications will become apparent to the skilled practitioner upon a study of the drawings, the specification and the following claims.

What is claimed is:

1. A tool for use during dual-bundle ligament reconstruction comprising:
a body including a distal portion and a proximal portion, said body having a cannulated longitudinal shaft, said cannulation defining a first axis;
a locating feature formed at said distal portion and defining a locating surface, said locating surface defining a second axis arranged at a predetermined orientation relative to said first axis; and
wherein a first locating instrument extending through a first bone tunnel is adapted to rest against said locating surface of said groove to operatively align said first axis at said predetermined orientation thereby identifying a drill axis for preparation of a second bone tunnel.

2. The tool of claim 1 wherein said locating feature is defined by a groove formed in said distal portion, said groove defining said locating surface.

3. The tool of claim 1 wherein said distal portion includes a distal engaging surface oriented at a non-orthogonal angle relative to said first axis.

4. The tool of claim 1 wherein said proximal portion includes a handle defining a second axis oriented at a non-orthogonal angle relative to said first axis.

5. The tool of claim 1 wherein said longitudinal shaft defines a first outer diameter and wherein said distal portion defines a second outer diameter, wherein said first outer diameter is less than said second outer diameter.

6. The tool of claim 5 wherein a tapered portion is defined between said distal portion and said longitudinal shaft.

7. The tool of claim 6 wherein said first locating instrument is adapted to pass into an anterior medial (AM) tunnel, and wherein a second locating instrument is adapted to pass through said cannulated shaft and into a posterior lateral (PL) tunnel.

8. A tool for use during dual-bundle ligament reconstruction comprising:
a first locating instrument defining a first longitudinal shaft portion having a first axis, a first reference head and a first distal tip portion;
a second locating instrument defining a second longitudinal shaft portion having a second axis, and a second reference head;
wherein said first locating instrument is adapted to be located into a first bone tunnel and wherein said second reference head of said second locating member is adapted to engage said first reference head to operatively align said second axis at a predetermined orientation thereby identifying a drill axis for preparation of a second bone tunnel.

9. The tool of claim 8 wherein said first and second locating instruments define a first and second handle on proximal portions of said first and second longitudinal shafts respectively.

10. The tool of claim 9 wherein said first and second handles are removably secured to said respective longitudinal shafts by way of a threadable fastener secured to threads formed on said proximal portions of said first and second longitudinal shafts respectively.

11. The tool of claim 10 wherein said first and second head portions define a width substantially equivalent to a diameter of said first and second bone tunnels.

12. The tool of claim 11 wherein said second locating instrument is adapted to pass through an anterolateral portal prepared in a knee.

13. The tool of claim 6 wherein said first locating instrument is adapted to pass into an anterior lateral (AL) tunnel, and wherein a second locating instrument is adapted to align coaxial to a posterior medial (PM) tunnel.

14. A method for drilling a bone for receipt of a first and second bundle, the method comprising:
drilling a first tunnel in the bone;
positioning a locating instrument into the first bone tunnel thereby defining a reference axis;
inserting a tool through a portal defined in surrounding tissue;
engaging the tool with the locating instrument thereby identifying a relative orientation between the reference axis and a drill axis for preparation of a second bone tunnel; and
drilling the second bone tunnel along the drill axis.

15. The method of claim 14 wherein inserting the tool through the portal includes inserting the tool through a medial portal in a knee.

16. The method of claim 15 wherein positioning the locating instrument into the first bone tunnel includes locating the locating instrument into an anterior medial (AM) tunnel and wherein the second bone tunnel defines a posterior lateral (PL) tunnel.

17. The method of claim 16 wherein engaging the tool with the locating instrument includes engaging the locating instrument against a groove defined in the engaging tool.

18. The method of claim 14 wherein inserting the tool through the portal includes inserting the tool through an anterior-lateral portal in a knee.

19. The method of claim 18 wherein engaging the tool with the locating instrument includes engaging a first reference head formed on the locating instrument with a second reference head formed on the tool to operatively align the reference axis to the drill axis for preparation of the second bone tunnel.

20. The method of claim 19 wherein the first and second reference heads each define a width equivalent to a reamer diameter adapted to form the respective first and second bone tunnels respectively, and wherein engaging the first reference head with the second reference head verifies a desired offset for the respective first and second bone tunnels.
